(19) United States
(12) Patent Application Publication
(10) Pub. No.: US 2026/0204715 A1
(43) Pub. Date: Jul. 16, 2026

(54) ENHANCED POWER SUPPLY FOR AN ORTHOPEDIC TOOL

(71) Applicant: Fidelis Partners, LLC, Cheyenne, WY (US)

(72) Inventor: Christopher PEDICINI, Brentwood, TN (US)

(73) Assignee: Fidelis Partners, LLC, Cheyenne, WY (US)

(21) Appl. No.: 19/451,329

(22) Filed: Jan. 16, 2026

Related U.S. Application Data
(60) Provisional application No. 63/746,074, filed on Jan. 16, 2025.

(51) Int. Cl. H01M 50/247; A61B 17/92; H01M 10/42

(52) U.S. Cl. CPC H01M 50/247; A61B 17/92; H01M 10/425; A61B 2017/922; H01M 2220/30

(57) ABSTRACT

An orthopedic tool may include a power supply configured to provide power to the orthopedic tool, a first energy storage element associated with the power supply, a second energy storage element associated with the power supply, and a structure. The structure may be configured to, when in a first configuration, electrically decouple the second energy storage element from the first energy storage element such that the power supply is configured to provide power at a first voltage associated with the first energy storage element, and when in a second configuration, electrically couple the second energy storage element to the first energy storage element such that the power supply is configured to provide power at a second voltage associated with the first energy storage element and the second energy storage element.

### CROSS-REFERENCE TO RELATED APPLICATION

This application claims the benefit of U.S. Provisional Application No. 63/746,074, filed Jan. 16, 2025, which is incorporated herein by reference in its entirety.

### BACKGROUND

Orthopedic tools are commonly used to deliver mechanical energy during procedures, such as by delivering mechanical energy to surgical instruments. Such orthopedic tools are powered by a power supply, such as a power supply that provides power to one or more components of the orthopedic tools.

### SUMMARY

In some aspects, the techniques described herein relate to an orthopedic tool, including: a power supply configured to provide power to the orthopedic tool; a first energy storage element associated with the power supply; a second energy storage element associated with the power supply; and a structure configured to: when in a first configuration, electrically decouple the second energy storage element from the first energy storage element such that the power supply is configured to provide power at a first voltage associated with the first energy storage element, and when in a second configuration, electrically couple the second energy storage element to the first energy storage element such that the power supply is configured to provide power at a second voltage associated with the first energy storage element and the second energy storage element.

In some aspects, the techniques described herein relate to a power supply for an orthopedic tool, including: a first energy storage element; a second energy storage element; and a structure configured to: when in a first configuration, electrically decouple the second energy storage element from the first energy storage element such that the power supply is configured to provide power at a first voltage associated with the first energy storage element, and when in a second configuration, electrically couple the second energy storage element to the first energy storage element such that the power supply is configured to provide power at a second voltage associated with the first energy storage element and the second energy storage element.

In some aspects, the techniques described herein relate to a system including: an orthopedic tool; and a power supply including a first energy storage element, a second energy storage element, and a structure configured to, when in a first configuration, electrically decouple the second energy storage element from the first energy storage element, and, when in a second configuration, electrically couple the second energy storage element to the first energy storage element, wherein, when the structure is in the first configuration, the power supply is configured to provide power at a first voltage associated with the first energy storage element, and when the structure is in the second configuration, the power supply is configured to provide power at a second voltage associated with the first energy storage element and the second energy storage element.

### BRIEF DESCRIPTION OF THE DRAWINGS

FIGS. 1A-1C are diagrams of an example associated with an enhanced power supply for an orthopedic tool.

### DETAILED DESCRIPTION OF EXAMPLE EMBODIMENTS

The following detailed description of example implementations refers to the accompanying drawings. The same reference numbers in different drawings may identify the same or similar elements.

Orthopedic procedures may require delivery of mechanical energy at different power levels at different stages of a procedure or across different types of procedures. In some cases, a higher level of power may be desirable to increase mechanical output, while in other situations a lower level of power may be desirable to provide greater control, reduce delivered energy, or accommodate procedural requirements.

Orthopedic tools are often powered by a battery, or other energy storage element, that provides a fixed output voltage, which may limit the ability to selectively vary operating power. In such arrangements, the operating power of the orthopedic tool is determined by the provided voltage and does not readily support selective variation of operating power based on procedural requirements.

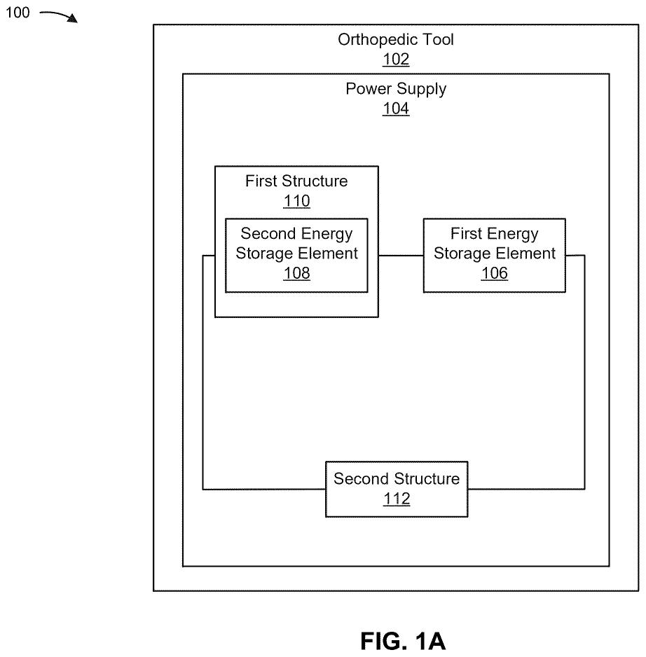

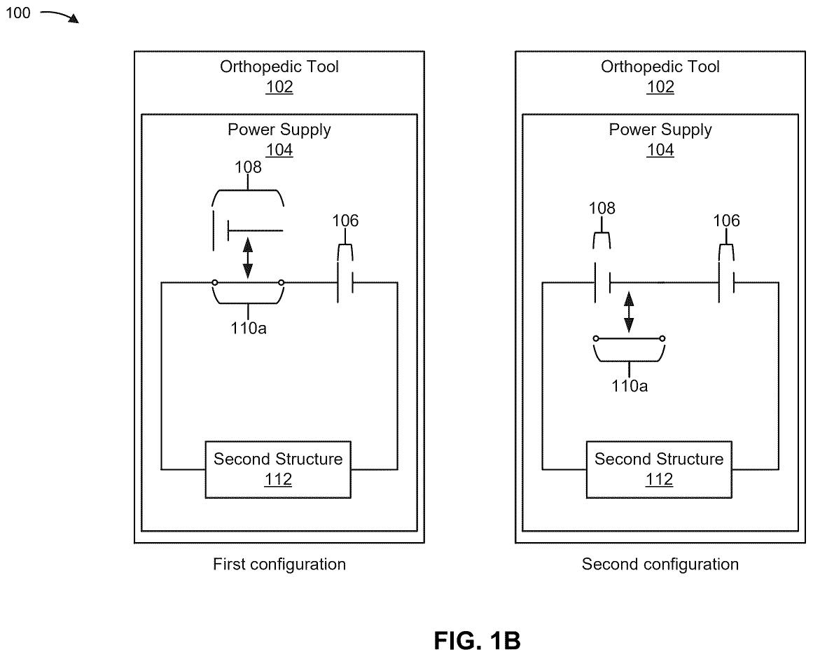

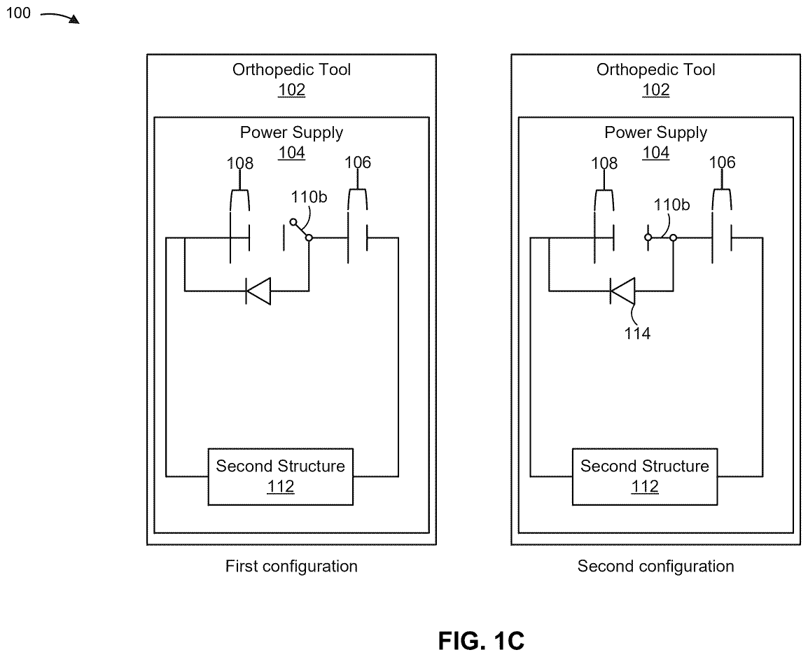

FIGS. 1A-1C are diagrams of an example associated with an enhanced power supply for an orthopedic tool. As shown in FIG. 1A, the example 100 includes an orthopedic tool 102 and a power supply 104.

In some implementations, the orthopedic tool 102 may be configured to be powered by the power supply 104. For example, the orthopedic tool 102 may be an orthopedic impactor, an orthopedic reamer, and/or an orthopedic saw, among other examples, configured to receive electrical power from the power supply 104, as described in more detail elsewhere herein.

In some implementations, the orthopedic tool 102 may include a first structure (e.g., shown as a first structure 110 in FIG. 1A) configured to interact with power (e.g., electrical power) provided by the power supply 104 by selectively influencing an electrical coupling between components of the power supply 104, as described in more detail elsewhere herein. For example, the first structure 110 may include one or more electrical, electronic, electromechanical, and/or mechanical components configured to selectively establish, inhibit, modify, and/or control a conductive path associated with the power supply 104, such as to control whether electrical power provided by the first energy storage element 106 and/or the second energy storage element 108 is combined or delivered in different configurations, among other examples. In some implementations, the first structure 110 may be physically integrated with, mounted to, or electrically associated with the power supply 104, as described in more detail elsewhere herein.

In some implementations, the power supply 104 may include a first energy storage element (e.g., shown as a first energy storage element 106 in FIG. 1A), such as a first battery, and a second energy storage element (e.g., shown as a second energy storage element 108 in FIG. 1A), such as a second battery. Accordingly, for example, the power supply 104 may be configured to provide power to the orthopedic tool 102 (e.g., to one or more components of the orthopedic tool 102 and/or one or more components associated with the orthopedic tool 102, among other examples) via the first energy storage element 106 and/or the second energy storage element 108, as described in more detail elsewhere herein.

In some implementations, the first structure 110 may be configured to control (e.g., selectively control) an electrical coupling between the first energy storage element 106 and the second energy storage element 108. For example, the first structure 110 may be configured to selectively establish or inhibit an electrically conductive path such that the second energy storage element 108 is electrically coupled to the first energy storage element 106 (e.g., the second energy storage element 108 may be electrically coupled in series with the first energy storage element 106 in some configurations) and is not electrically coupled to the first energy storage element 106 in other configurations (e.g., the second energy storage element 108 may not be electrically coupled in series with the first energy storage element 106 in other configurations), as described in more detail elsewhere herein.

Accordingly, in some implementations, the first structure 110 may be configured to form (e.g., either directly or indirectly) part of a conductive path of the power supply 104, such as part of a selectively configurable electrically conductive path of the power supply 104. For example, the first structure 110 may be associated with multiple configurations, such as a first configuration (e.g., a coupled or installed configuration) and a second configuration (e.g., a decoupled or uninstalled configuration).

In the first configuration, the first structure 110 may be configured to provide a conductive path (e.g., an electrically conductive path) that bypasses the second energy storage element 108 such that the second energy storage element 108 is not electrically coupled to the first energy storage element 106 (e.g., the second energy storage element 108 may not be electrically coupled in series with the first energy storage element 106 when the first structure 110 is in the configuration). Accordingly, when the first structure 110 is in the first configuration, the power supply 104 may be configured to provide power (e.g., electrical power) to the orthopedic tool 102 at a first voltage (e.g., a first operating voltage) corresponding to a voltage associated with the first energy storage element 106.

In the second configuration, the first structure 110 may be configured such that the conductive path that bypasses the second energy storage element 108 is not provided (e.g., the first structure 110 may be configured to refrain from providing the electrically conductive path that bypasses the second energy storage element 108 when in the second configuration) such that the second energy storage element 108 is electrically coupled to the first energy storage element 106 (e.g., the second energy storage element 108 may be electrically coupled in series with the first energy storage element 106 when the first structure 110 is in the second configuration).

Accordingly, when the first structure 110 is in the second configuration, the power supply 104 may be configured to provide power (e.g., electrical power) to the orthopedic tool 102 at a second voltage (e.g., a second operating voltage of the orthopedic tool 102) corresponding to a voltage associated with the first energy storage element 106 and the second energy storage element 108 (e.g., a combined voltage associated with the first energy storage element 106 and the second energy storage element 108).

As an example, the first structure 110 may be implemented as a shunt configured for operation between the first configuration and the second configuration (e.g., shown as a shunt 110a in FIG. 1B in the first configuration and the second configuration). As another example, the first structure 110 may be implemented as a switch configured for operation between the first configuration and the second configuration (e.g., shown as a switch 110b in the first configuration and the second configuration in FIG. 1C).

Although the electrical coupling between the first energy storage element 106 and the second energy storage element 108 is described herein as being controlled by the first structure 110, such coupling may be controlled in other manners in some other implementations. For example, the coupling may be controlled by one or more additional and/or alternative structures configured to selectively establish, inhibit, and/or modify a conductive path (e.g., an electrically conductive path) between the first energy storage element 106 and the second energy storage element 108, such as a relay, a jumper, a solid-state device, a diode, a transistor, and/or a mechanical contact, among other examples. In some implementations, such control may be based on a configuration associated with the power supply 104, insertion and/or removal of an energy storage element, actuation of a control element, detection of a condition (e.g., an electrical condition), and/or interaction with another component of the orthopedic tool 102, among other examples. In some implementations, such control may be implemented through hardware configuration and/or electrical topology (e.g., without requiring modification of software associated with the orthopedic tool 102), while in some other implementations software may be used to facilitate such control.

In some implementations, the orthopedic tool 102 may include a second structure (e.g., shown as a second structure 112 in FIG. 1A), configured to receive and/or use power (e.g., electrical power) provided by the power supply 104. For example, the second structure 112 may include one or more electrical, electronic, and/or electromechanical components configured to distribute, condition, regulate, and/or control power provided by the power supply 104 to one or more functional components of the orthopedic tool 102, such as a motor, actuator, controller, sensor, and/or a load (e.g., an electrical load), among other examples. Accordingly, in some implementations, the second structure 112 may be implemented as, or may include, a printed circuit board, a power management circuit, a motor drive circuit, and/or a control circuit, among other examples.

In some implementations, the power supply 104 may include (e.g., optionally include) one or more components configured to facilitate isolation (e.g., shown as a diode 114 in FIG. 1C), such as by being configured to influence current flow between components of the power supply 104, among other examples. As an example, the one or more components may be configured to inhibit current flow, such as reverse current flow between the first energy storage element 106 and the second energy storage element 108, cross-charging between the first energy storage element 106 and the second energy storage element 108, and/or current flow under one or more conditions (e.g., one or more operating conditions), among other examples.

Accordingly, in some implementations, the one or more components may include a diode, a diode circuit, and/or a transistor-based arrangement, among other examples, configured to control current flow (e.g., although the inclusion of the one or more components may be optional and is not required for electrical coupling between the first energy storage element 106 and the second energy storage element 108).

As indicated above, FIGS. 1A-1C are provided as examples. Other examples may differ from what is described with regard to FIGS. 1A-1C. The number and arrangement of the various components shown in FIGS. 1A-1C are provided as examples. In practice, there may be additional components, fewer components, different components, or differently arranged components than those shown in FIGS. 1A-1C. Additionally, or alternatively, a set of components (e.g., one or more components) shown in FIGS. 1A-1C may perform one or more functions described as being performed by another set of components shown in FIGS. 1A-1C.

As used herein, satisfying a threshold may, depending on the context, refer to a value being greater than the threshold, greater than or equal to the threshold, less than the threshold, less than or equal to the threshold, equal to the threshold, not equal to the threshold, or the like.

Even though particular combinations of features are recited in the claims and/or disclosed in the specification, these combinations are not intended to limit the disclosure of various implementations. In fact, many of these features may be combined in ways not specifically recited in the claims and/or disclosed in the specification. Although each dependent claim listed below may directly depend on only one claim, the disclosure of various implementations includes each dependent claim in combination with every other claim in the claim set. As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiple of the same item.

No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, or a combination of related and unrelated items), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

In the preceding specification, various example embodiments have been described with reference to the accompanying drawings. It will, however, be evident that various modifications and changes may be made thereto, and additional embodiments may be implemented, without departing from the broader scope of the invention as set forth in the claims that follow. The specification and drawings are accordingly to be regarded in an illustrative rather than restrictive sense.

What is claimed is:

1. An orthopedic tool, comprising:
a power supply configured to provide power to the orthopedic tool;
a first energy storage element associated with the power supply;
a second energy storage element associated with the power supply; and
a structure configured to:
when in a first configuration, electrically decouple the second energy storage element from the first energy storage element such that the power supply is configured to provide power at a first voltage associated with the first energy storage element, and
when in a second configuration, electrically couple the second energy storage element to the first energy storage element such that the power supply is configured to provide power at a second voltage associated with the first energy storage element and the second energy storage element.

2. The orthopedic tool of claim 1, wherein the first configuration is associated with a first operating power of the orthopedic tool based on operation at the first voltage, and the second configuration is associated with a second operating power of the orthopedic tool based on operation at the second voltage that is greater than the first operating power.

3. The orthopedic tool of claim 1, wherein the structure is configured such that, in the second configuration, the second energy storage element is electrically coupled in series with the first energy storage element.

4. The orthopedic tool of claim 1, wherein the first voltage and the second voltage correspond to different power output levels of the orthopedic tool.

5. The orthopedic tool of claim 1, wherein power available to the orthopedic tool when the second energy storage element is electrically coupled to the first energy storage element is increased relative to power available to the orthopedic tool when the second energy storage element is electrically decoupled from the first energy storage element.

6. The orthopedic tool of claim 1, wherein the second voltage is greater than the first voltage.

7. The orthopedic tool of claim 1, wherein the structure includes a shunt, and wherein installation of the shunt corresponds to the first configuration and removal of the shunt corresponds to the second configuration.

8. The orthopedic tool of claim 1, wherein the structure includes a switch, and wherein the structure is configured to transition between the first configuration and the second configuration in response to actuation or deactuation of the switch.

9. The orthopedic tool of claim 1, wherein the orthopedic tool includes an impactor configured to provide a first impact energy when the structure is in the first configuration and a second impact energy when the structure is in the second configuration that is greater than the first impact energy.

10. The orthopedic tool of claim 1, wherein at least one of the first configuration or the second configuration is associated with a requirement related to a procedure associated with the orthopedic tool.

11. The orthopedic tool of claim 1, wherein the orthopedic tool is configured to transition between the first configuration and the second configuration during a procedure.

12. A power supply for an orthopedic tool, comprising:
a first energy storage element;
a second energy storage element; and
a structure configured to:
when in a first configuration, electrically decouple the second energy storage element from the first energy storage element such that the power supply is configured to provide power at a first voltage associated with the first energy storage element, and
when in a second configuration, electrically couple the second energy storage element to the first energy storage element such that the power supply is configured to provide power at a second voltage associated with the first energy storage element and the second energy storage element.

13. The power supply of claim 12, wherein the first configuration is associated with a first operating power of the orthopedic tool based on operation at the first voltage, and the second configuration is associated with a second operating power of the orthopedic tool based on operation at the second voltage that is greater than the first operating power.

14. The power supply of claim 12, wherein the structure is configured such that, in the second configuration, the second energy storage element is electrically coupled in series with the first energy storage element.

15. The power supply of claim 12, wherein the first voltage and the second voltage correspond to different power output levels of the orthopedic tool.

16. The power supply of claim 12, wherein power available to the orthopedic tool when the second energy storage element is electrically coupled to the first energy storage element is increased relative to power available to the orthopedic tool when the second energy storage element is electrically decoupled from the first energy storage element.

17. A system comprising:
an orthopedic tool; and
a power supply including a first energy storage element, a second energy storage element, and a structure configured to, when in a first configuration, electrically decouple the second energy storage element from the first energy storage element, and, when in a second configuration, electrically couple the second energy storage element to the first energy storage element,
wherein, when the structure is in the first configuration, the power supply is configured to provide power at a first voltage associated with the first energy storage element, and when the structure is in the second configuration, the power supply is configured to provide power at a second voltage associated with the first energy storage element and the second energy storage element.

18. The system of claim 17, wherein the first configuration is associated with a first operating power of the orthopedic tool based on operation at the first voltage, and the second configuration is associated with a second operating power of the orthopedic tool based on operation at the second voltage that is greater than the first operating power.

19. The system of claim 17, wherein the structure is configured such that, in the second configuration, the second energy storage element is electrically coupled in series with the first energy storage element.

20. The system of claim 17, wherein the first voltage and the second voltage correspond to different power output levels of the orthopedic tool.
